(19) United States
(12) Patent Application Publication
(10) Pub. No.: US 2009/0041858 A1
(43) Pub. Date: Feb. 12, 2009

(54) Haemostatic Compositions Thickened by Polymers Containing Nitrogen Moieties

(75) Inventor: Steven D. Jensen, South Jordan, UT (US)

Correspondence Address: GEOFFREY E. DOBBIN, PATENT ATTORNEY, 4278 SOUTH 6220 WEST, WEST VALLEY CITY, UT, 84128-6501, US

(73) Assignee: CAO GROUP, INC., West Jordan, UT (US)

(21) Appl. No.: 11/834,455

(22) Filed: Aug. 6, 2007

(51) Int. Cl. A61K 33/26; A61K 33/06; A61P 7/00

(52) U.S. Cl. 424/647; 424/648; 424/682; 424/685

(57) ABSTRACT

The present invention is the development of haemostatic compositions that use nitrogen containing organic polymers in combination with haemostatic agents to create a gel form. Compositions utilizing the polymers exhibit relatively lower concentrations of haemostatic agents, drawing benefits from such lower concentrations as they limit unwanted side effects of the haemostatic agents. Preferred compositions utilize Poly(N-vinylpyrrolidone), Poly(2-ethyl-2-oxazoline) as thickening agents and ferric sulfate as an active ingredient.

### FIELD OF THE INVENTION

The present invention relates to the field of haemostatic compositions and more particularly relates to haemostatic compositions containing nitrogen moieties and ferric and/or aluminum compounds.

### BACKGROUND OF THE INVENTION

In dentistry or any health care field including veterinary medicine, there is a need to control bleeding during routine procedures and surgical operations. The ability to control or manage bleeding allows for a clean operating environment. Blood is a nuisance during surgeries because it coats and covers the work area and generally makes a mess.

The body of course has developed its own mechanism to stop bleeding. The oldest way to stop bleeding is to apply pressure or a bandage and, over time, the body will form a temporary clot to staunch the bleeding and eventually heal itself. All haemostatic agents are designed to complement and/or speed up this process, because in many cases the body cannot staunch all bleeding without intervention. For example, a cut artery left unattended will result in death as the body has no mechanism to control this level of bleeding. This is why we apply pressure or a bandage to a more severe wound to keep the blood from escaping so that the body can eventually form a clot by itself. That is why it is so important to keep blood in the body. Haemostatic compounds are designed to control bleeding beyond the ordinary physiological processes of the body.

Haemostatic compounds have therefore been developed and work in one of three ways:
1. Coagulation or clotting compounds: Compounds that initiate the clotting factor and cause the blood to coagulate. Some compounds that exhibit this type of haemostasis are the salts of iron. Ferric and ferrous sulfate and ferric and ferrous chloride.
2. Local astringents: Compounds that constrict the blood vessels locally whereby slowing the flow of blood, similar to the action of antiperspirant which constricts the flow of sweat. Aluminum chloride and sulfate are two such compounds.
3. Vasoconstrictors: Compounds, usually organic, that work systemically or locally to constrict the flow of blood. Examples are epinephrine and salts of epinephrine.

In dentistry the control of bleeding is paramount because blood can interfere in various procedures. Blood that oozes onto a prepared tooth while applying an adhesive allows blood and adhesive to be combined for detrimental effects to the adhesive and aesthetics. Also, blood oozing from the gums while taking an impression for a crown will interfere with the impression material, providing a bad impression, which will correlate to a poor fitting crown. In order for a dentist to function, blood must be able to be controlled.

Many current haemostatic products on the market are aqueous compositions that have the viscosity of water. These products are effective, but flow all over the mouth. There is little control of a liquid haemostatic once it hits the moist mouth as it tends to run in all directions. In order to better control haemostatic compositions in the next generation of haemostatic agents, manufacturers have added thickeners to increase the viscosity of their products. Higher viscosity gels are an improvement, because they limit the flow of the composition and make it easier for the dentist to place the product. Comparisons in efficacy between gel formulations and aqueous formulations are difficult as aqueous formulations (having a viscosity close to water, about 1-5 centipoise) tend to quickly disperse and dilute with bodily fluids at a faster rate than gel formulations. As such, their efficacy is diminished quickly. This is one reason that a gel formulation is superior.

The most current generation of products are thickened by inorganic silica powders. However, as the thickening increases the haemostatic effectiveness generally decreases. In order to get the same effectiveness as a liquid haemostatic agent you have to increase the concentration of the haemostatic agent. Unfortunately, most haemostatic agents are aggressive salts which in higher concentrations precipitate, coagulate, or otherwise destroy thickening polymers (thus the use of inorganic silica). Therefore, if one was to thicken haemostatic compositions in the past, they had to trade off effectiveness with viscosity until a tolerable balance was reached.

High concentrations of ferric sulfate, as an example, though effective in staunching bleeding, are caustic and cause sloughing and blackening of soft tissues. High ferric sulfate concentrations that are close to the saturation point tend to precipitate out fine elemental iron into the composition. This free elemental iron tends to stay behind after treatment because it is very hard to wash off. Elemental iron is insoluble in water and cannot be dissolved, therefore it is left behind to cause those black stains in between restorations and the tooth. This black staining of restorations is common among restoration problems. The dentist must go through a cleaning regime in order to remove residual or precipitated iron from the prep prior to restoration. It has also been shown that residual ferric sulfate interferes with restorative agents, particularly bonding adhesives.

The present invention represents a departure from the prior art in that the composition of the present invention allows for the use of less ferric/aluminum compounds by weight than prior art compositions, increasing the available viscosity levels while not impeding effectiveness. It also allows for the use of readily available, inexpensive organic thickeners.

### SUMMARY OF THE INVENTION

In view of the foregoing disadvantages inherent in the known types of haemostatic compounds, this invention provides improved haemostatic compounds with greater viscosity and less active ingredient. As such, the present invention's general purpose is to provide a new and improved haemostatic compounds that are easy to manufacture and store, relatively inexpensive to manufacture and more readily have desired viscosity and effectiveness.

To accomplish these objectives, the haemostatic compounds according to the present invention comprise novel nitrogen containing organic polymers that are compatible with these haemostatic agents and form stable uniform gels with them. The best polymers we have experimented with are Poly-2-ethyl-2-oxazoline (“Oxazoline”) and N-Vinyl-2-pyrrolidone (“PVP”). What we have found is that they are excellent gelling agents that provide optimal viscosities. It is surprising that these two polymers are capable of forming a gel with these haemostatics, as most thickeners are destroyed by these aggressive compounds. The biggest surprise is that they actually do the opposite of silica—that is they do not diminish the effects of the haemostatic agents. We can get haemostatic power similar to an aqueous product having 20% or more haemostatic agent by weight as compared to a 10% product using the teachings of this Specification. Having less active chemical is beneficial in many ways as there is less harm to soft tissue, less staining, easier rinsing of the area after a procedure is finished, less required dose and, particularly in dentistry, better taste (as most haemostatic chemicals taste bitter).

The more important features of the invention have thus been outlined in order that the more detailed description that follows may be better understood and in order that the present contribution to the art may better be appreciated. Additional features of the invention will be described hereinafter and will form the subject matter of the claims that follow.

Many objects of this invention will appear from the following description and appended claims, reference being made to the accompanying drawings forming a part of this specification wherein like reference characters designate corresponding parts in the several views.

Before explaining at least one embodiment of the invention in detail, it is to be understood that the invention is not limited in its application to the details of construction and the arrangements of the components set forth in the following description or illustrated in the drawings. The invention is capable of other embodiments and of being practiced and carried out in various ways. Also it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### DETAILED DESCRIPTION OF THE PREFERRED EMBODIMENT

With reference now to the drawings, the preferred embodiments of the haemostatic compounds are herein described. It should be noted that the articles “a”, “an”, and “the”, as used in this specification, include plural referents unless the content clearly dictates otherwise.

Many different combinations and compositions have been developed in accordance with this invention. This Specification will use the example of ferric sulfate in dentistry for illustrative purposes, as it is generally accepted as a good haemostatic agent to control operative bleeding for many procedures. It should, of course, be realized that these compounds are useful in any situation where blood loss is to be controlled beyond a body's normal capacity, including other fields of medicine and surgery, veterinary medicine and emergency care. It should also be noted that there are other haemostatic agents, such as ferrous sulfate and ferric and ferrous chloride, aluminum compounds and other salts that are also haemostatics and could be used in this invention. There are other haemostatic compositions which could be used in this invention, but are not considered ideal. One such haemostatic is ferric sub-sulfate, which is extremely caustic and causes sloughing of soft tissues. Another is epinephrine, which is slow acting and causes systemic side-effects (i.e. increased heart rate). The most advantageous haemostatic compounds, in our experience, are: ferric and ferrous sulfate, ferric and ferrous chloride, aluminum chloride and aluminum sulfate.

In order for ferric sulfate to work effectively in cases where the patient is a heavy bleeder, it requires a higher concentration to cause homeostasis. To effectively cover all varieties of humans, many practitioners use an aqueous liquid concentration of ferric sulfate so that the final concentration contains over 5.5% iron ion by weight, or over 20% ferric sulfate by weight. We can get similar effectiveness with PVP and Oxazoline compositions at 2.79% iron ion or 10% ferric sulfate.

Because of the low concentration the final product does not approach the saturation point of ferric sulfate, therefore all of the iron in the system stays water-soluble and therefore easy to wash off and causes less staining. There is also less residual ferric sulfate to interfere with bonding agents.

The benefits of this invention are found when using polymers containing nitrogen. It is suspected that the nitrogen groups are acting as a mild cat-ion, tying up negatively charged sulfate anions making the ferric ions less hindered and therefore more active. The best mode ingredients would be those nitrogen containing polymers that are:
1. water soluble
2. non-toxic
3. ionically balanced such that they increase the efficiency or at least not hinder the efficiency of haemostatic agents.
   PVP and Oxazoline are preferred as they effectively thicken the compositions while not hindering the efficiency of the haemostatic agents, at least not any more than prior art thickeners.

For haemostatic agents the best mode ingredients would be those that are:
1. water soluble
2. non-toxic, and
3. able to effectively stop bleeding on contact.
   Preferred haemostatic agents include: ferric sulfate, ferrous sulfate, ferric chloride, ferrous chloride, aluminum chloride, aluminum sulfate, epinephrine and salts of epinephrine. Compositions may be made of a basic combination of the haemostatic agent, the polymer and a solvent. Water is an ideal and preferred solvent, though others such as ethanol, propylene glycol, glycerin and combinations of the same may be used. The percentage of haemostatic agent by weight should have a maximum range between 0.1%-65%. Ideally, the range should be between 1 and 20%, though ranges between 0.5% and 35% also exhibit greater workability. To obtain a desired thickness, then, the composition merely needs to balance solvent and polymer with the remaining percentage. Viscosity of the eventual gels may range between 500 to 200,000 centipoise, with a range of 1,000 to 60,000 centipoise being preferred.

Although the present invention has been described with reference to preferred embodiments, numerous modifications and variations can be made and still the result will come within the scope of the invention. No limitation with respect to the specific embodiments disclosed herein is intended or should be inferred.

What is claimed is:

1. A haemostatic composition comprising:
a. A nitrogen containing organic polymer; and
b. A haemostatic agent, the haemostatic agent being selected from the group of haemostatic agents consisting of: ferric sulfate, ferrous sulfate, ferric chloride, ferrous chloride, aluminum chloride, and aluminum sulfate.

2. The haemostatic composition of claim 1, the polymer being selected from the group of polymers consisting of: Poly(N-vinylpyrrolidone) and Poly(2-ethyl-2-oxazoline).

3. The haemostatic composition of claim 1, the percentage weight of the haemostatic agent being between 0.1 and 65% of the total composition.

4. The haemostatic composition of claim 1, the percentage weight of the haemostatic agent being between 0.5 and 35% of the total composition.

5. The haemostatic composition of claim 1, the percentage weight of the haemostatic agent being between 1 and 20% of the total composition.

6. The haemostatic composition of claim 1, the composition being a gel with a viscosity between 500 and 200,000 centipoise.

7. The haemostatic composition of claim 1, the composition being a gel with a viscosity between 1,000 and 60,000 centipoise.

8. The haemostatic composition of claim 1, the haemostatic agent being ferric sulfate and the polymer being selected from the group of polymers consisting of Poly(N-vinylpyrrolidone) and Poly(2-ethyl-2-oxazoline).

9. The haemostatic composition of claim 8, the percentage weight of the haemostatic agent being between 0.1 and 65% of the total composition.

10. The haemostatic composition of claim 9, the composition being a gel with a viscosity between 500 and 200,000 centipoise.

11. The haemostatic composition of claim 9, the composition being a gel with a viscosity between 1,000 and 60,000 centipoise.

12. The haemostatic composition of claim 8, the percentage weight of the haemostatic agent being between 0.5 and 35% of the total composition.

13. The haemostatic composition of claim 12, the composition being a gel with a viscosity between 500 and 200,000 centipoise.

14. The haemostatic composition of claim 12, the composition being a gel with a viscosity between 1,000 and 60,000 centipoise.

15. The haemostatic composition of claim 8, the percentage weight of the haemostatic agent being between 1 and 20% of the total composition.

16. The haemostatic composition of claim 15, the composition being a gel with a viscosity between 500 and 200,000 centipoise.

17. The haemostatic composition of claim 15, the composition being a gel with a viscosity between 1,000 and 60,000 centipoise.
